(19) United States
(12) Patent Application Publication
(10) Pub. No.: US 2022/0387288 A1
(43) Pub. Date: Dec. 8, 2022

(54) CROSSLINKED POLYMER LATEX AND LOW-PH TRANSPARENT AQUEOUS COMPOSITION FOR HAIR OR SKIN CARE

(71) Applicant: SPCM SA, Andrezieux Boutheon (FR)

(72) Inventors: Frédéric BLONDEL, Andrezieux Boutheon (FR); Thomas BOURSIER, Andrezieux Boutheon (FR)

(73) Assignee: SPCM SA, Andrezieux Boutheon (FR)

(21) Appl. No.: 17/756,071

(22) PCT Filed: Dec. 14, 2020
(86) PCT No.: PCT/FR2020/052433

§ 371 Date: May 16, 2022

(30) Foreign Application Priority Data
Dec. 23, 2019 (FR) FR1915453

(51) Int. Cl. A61K 8/81; A61Q 19/00; A61Q 5/00

(52) U.S. Cl. CPC A61K 8/8152; A61K 8/8158; A61Q 19/00; A61Q 5/00

(57) ABSTRACT

The present invention relates to a latex comprising a crosslinked polymer, the polymer comprising 20 to 45 wt % of (meth)acrylic acid monomer units, 35 to 78 wt % of methylacrylate and/or ethylacrylate monomer units, 0 to 30 wt % of monomer units of at least one linear alkyl acrylate, the linear alkyl comprising 3 to 9 carbon atoms, characterized in that the polymer is crosslinked with N-methylenebisacrylamide. The latex according to the invention helps thicken a composition for hair and/or skin care having a pH of between 3.5 and 5.5.

### TECHNICAL FIELD OF THE INVENTION

The present invention relates to new latices of crosslinked polymers and their use for formulating generally low-pH transparent aqueous compositions for hair and/or skin care. The invention also relates to a low-pH transparent composition for hair and/or skin care comprising at least one latex according to the invention. The new latices are mainly used as rheology modifiers.

### PRIOR STATE OF THE ART

Aqueous hair and skin care compositions contain surfactants and require the use of preservatives. Many preservatives are listed as endocrine disruptors. Consequently, formulators are increasingly seeking to remove them from these compositions or to replace them with molecules which are still effective, but which do not present any danger to health.

Organic salts such as potassium sorbate and sodium benzoate meet both of these criteria. However, only the acid form of these salts is effective and it is therefore necessary to formulate the compositions at a pH below 5.5 in order for them to be effective. Furthermore, since the pH of the skin is close to 4.5-5, depending on the individual, the acid formulations around these values have the additional advantage of being less aggressive for the skin.

First generation rheology modifiers were created to provide viscosity, clarity and suspensivity in surfactant formulations at pH 6-6.5 and including paraben, hydantoin, or phenoxyethanol-type preservatives, listed as endocrine disruptors.

Therefore, the formulators substituted these preservatives with organic salts, thus reducing the pH of the compositions. However, the use of first-generation rheology modifiers at a more acidic pH (less than 5.5) significantly reduces the transparency of the compositions, which is detrimental for the good marketing of the compositions.

Document US 2013/0115185 discloses the use of a rheology modifier composed of a non-crosslinked core composed of methacrylic acid, a (meth)acrylate of (hydroxy)alkyl and a third monomer, associated with a crosslinked polymer ring composed of the same monomers. However, it appears difficult to combine both good rheological and optical properties in this way because the non-crosslinked core does not provide suspensivity. The polymers are crosslinked with trimethylolpropane triacrylate (TMPTA) and trimethylolpropane diallylether (TMPDAE).

Document WO 2016/102790 discloses a rheology modifier consisting of a multiphase polymer composed of a polymer 1 and a polymer 2, the composition of the two polymers optionally differing. Both polymers are crosslinked with trimethylolpropane triacrylate (TMPTA), and contain a specific associative monomer.

Document WO 2015/158668 discloses a rheology modifier and, more specifically, the use of a crosslinked polymer composed of methacrylic acid, alkyl acrylate, 2-acrylamido-2-methylpropane sulfonic acid (ATBS) and a specific hydrophobic monomer.

Document CN 105 968 251 discloses a pH-sensitive material and its use in the medical field, for example a crosslinked polymer of 50% by weight of acrylic acid and 50% by weight of ethyl acrylate. This document does not relate to cosmetic or pharmaceutical compositions.

Document EP 1 270 617 discloses a copolymer of an acid monomer and an ethoxylated monomer having a C31 or more hydrophobic fatty chain.

Document FR 2961815 discloses ATBS and acrylic acid polymers and their use for reducing the amount of air introduced into concrete formulations.

Therefore, despite these technological developments, there is a real need to develop new crosslinked polymer latices which make it possible to obtain an aqueous composition having suitable rheological properties and excellent transparency, including at acidic pH (in particular from 3.5 to 5.5).

The present invention makes it possible to solve these problems by proposing a latex comprising polymers of specific composition, the polymers being crosslinked with N-methylene-bis-acrylamide. The latices according to the invention offer a better compromise in terms of rheological performance and transparency, including at low pH (in particular from 3.5 to 5.5).

### DISCLOSURE OF THE INVENTION

The subject matter of the present invention is a latex comprising a crosslinked polymer, said polymer comprising:

from 20 to 45% by weight of monomer unit of (meth)acrylic acid;

from 35 to 78% by weight of monomer unit of methyl acrylate and/or ethyl acrylate;

from 0 to 30% by weight of monomer unit of at least one linear alkyl acrylate, the linear alkyl comprising 3 to 9 carbon atoms,

the sum of the monomer units being equal to 100% by weight.

This polymer is crosslinked with N-methylene-bis-acrylamide and is free of monomer unit having an alkyl chain of at least 10 carbon atoms, and monomer unit of type —(CHR¹—CHR²—O)— with R¹ and R² being, independently of each other, a hydrogen atom or an alkyl group.

The alkyl group of R¹ and R² generally contains between 1 and 50 carbon atoms.

The weight percentages of monomer units are expressed relative to the total weight of monomer units of the crosslinked polymer. The amount of crosslinking agent is not taken into account to determine these percentages.

“Methyl acrylate and/or ethyl acrylate”, is understood to mean either methyl acrylate, or ethyl acrylate, or methyl acrylate and ethyl acrylate. “(Meth)acrylic acid” is understood to mean either acrylic acid, or methacrylic acid, or acrylic acid and methacrylic acid.

The particular choice of N-methylene-bis-acrylamide (MBA) as crosslinking agent makes it possible to obtain an excellent compromise in terms of rheological properties, including at pH between 3.5 and 5.5, while having a very high transparency level.

Other crosslinking agents may be used in addition to the MBA, but in a preferred mode, the only crosslinking agent used to crosslink the polymer of the invention is N-methylene-bis-acrylamide (MBA). In another preferred mode, the crosslinking agents TMPTA or TMPDAE are not used to crosslink the polymer of the invention.

The presence in the polymer of a monomer unit having an alkyl chain of at least 10 carbon atoms, or of a monomer unit of the type —(CHR¹—CHR²—O)— with R¹ and R² being, independently of each other, a hydrogen atom or an alkyl group, is undesirable because these compounds are often difficult to source, or difficult to synthesize. They are not especially cost-effective and do not offer the best compromise between rheological property and clarity.

Crosslinked Polymer

The crosslinked polymer, present in the latex according to the invention, is crosslinked with an amount of N-methylene-bis-acrylamide advantageously between 500 and 5000 ppm by weight relative to the total weight of monomers, preferably between 1000 and 4000 ppm, more preferentially between 1500 and 3000 ppm, and even more preferentially between 1700 and 2500 ppm.

Methacrylic acid is advantageously preferred to acrylic acid.

Ethyl acrylate is advantageously preferred to methyl acrylate.

According to a preferred embodiment, the linear alkyl acrylate, the linear alkyl comprising 3 to 9 carbon atoms (CₙH₂ₙ₊₁ with n=1 to 9), is chosen from butyl acrylate, ethylhexyl acrylate and mixtures thereof, and is preferably butyl acrylate.

Advantageously, the polymer of the latex according to the invention is free of monomer unit having a hydrocarbon chain of at least 10 carbon atoms, this hydrocarbon chain being linear or branched, comprising at least one unsaturation, and having or not having cyclic groups.

The amount of (meth)acrylic acid monomer unit is preferably between 25 and 40% by weight, more preferably between 25 and 37% by weight.

Preferably, the amount of acrylic acid monomer unit is less than 20% by weight, preferably less than 15%, more preferably less than 10%, and more preferably less than 5%, the complement to 20%-45% by weight of monomer unit of (meth)acrylic acid being monomer units of methacrylic acid.

The polymer is preferably free of monomeric acrylic acid unit.

The amount of methylacrylate and/or ethylacrylate monomer unit is preferably between 40 and 75% by weight, more preferably between 45 and 70% by weight.

The amount of linear alkyl acrylate monomer unit, the linear alkyl comprising 3 to 9 carbon atoms, is preferably between 1 and 25% by weight, preferably at least 2%, more preferably at least 5% by weight.

The polymer is preferably free of strong acid monomer unit, more preferably free of 2-acrylamido-2-methylpropanesulfonic acid monomer unit (acronym ATBS, also called acrylamido-terbutyl sulfonic acid).

The amounts of monomer(s) can be adjusted by a person skilled in the art so that the sum of the amounts of monomers is equal to 100% by weight during the preparation of the crosslinked polymer according to the invention.

Latex

Originally, the term latex referred to the white substance produced by certain plants and secreted to defend themselves. The transformation of this natural material into rubber quickly developed at the end of the 19th century. Today, the term “latex” refers to any type of dispersion of hydrophobic or water-insoluble polymers in an aqueous solution. The dispersion of the latex particles is ensured by surfactant molecules at the interface, making it possible to reduce the interfacial tension. Synthetic latices are now widely used in various industries (tires, paints, adhesives, etc.).

The latex according to the invention preferably comprises between 20 and 40% by weight, more preferably between 25 and 35% by weight of crosslinked polymer. It preferably comprises between 60 and 80% by weight, preferably between 65 and 75% by weight of water. Furthermore, the latex preferably comprises between 0.1 and 2% of surfactant compounds and/or post-treatment compounds. For example, post-treatment compounds are initiators or preservatives. These weight percentages are expressed relative to the weight of the latex.

Latex Preparation Process

The latex according to the invention is prepared according to techniques known to a person skilled in the art. The latter will know how to adjust the preparation and polymerization parameters to obtain the latex according to the invention, while respecting the conditions of monomeric compositions and of crosslinking agent of the invention.

Generally, a latex is prepared as follows. Water and a first surfactant are introduced in a reactor, advantageously heated. A pre-emulsion containing water, the monomers to be polymerized, a second surfactant and the crosslinking agent, and an initiator solution are prepared at the same time. Once the reactor is at the desired temperature, the pre-emulsion and the initiator solution are added simultaneously to the reactor at the desired rate. The polymerization as well as the emulsification take place. After complete addition of the pre-emulsion and the solution, and complete reaction, a latex is obtained which can be filtered.

A person skilled in the art will know how to choose the appropriate surfactants, advantageously from oil-in-water surfactants, advantageously sodium dodecyl sulphate. Furthermore, the first and the second surfactant of the latex are advantageously identical.

Composition for Hair and/or Skin Care

The invention also relates to a hair and/or skin care composition comprising at least one latex according to the invention as described previously. The composition may be a cosmetic or pharmaceutical composition. By way of example, the composition is a shampoo composition, or a skin cleansing composition, or a shower gel composition. The composition according to the invention is generally transparent.

The composition preferably comprises an amount of latex of between 2 and 20% by weight relative to the total weight of the composition. Advantageously, it comprises an amount of crosslinked polymer of between 0.4 and 8% by weight relative to the total weight of the composition.

In addition to the latex surfactants, the composition for hair and/or skin care according to the invention generally comprises at least one surfactant, preferably at least two surfactants, which will provide the cleansing, even foaming, character to the composition. These surfactants are generally chosen from nonionic surfactants, anionic surfactants, cationic surfactants, amphoteric surfactants and zwitterionic surfactants. Preferably, the first surfactant has an anionic character, while the second surfactant has a zwitterionic character.

The composition according to the invention preferably comprises:

the latex according to the invention;

at least a first surfactant;

at least a second surfactant different from the first surfactant;

optionally at least one silicone agent;

optionally at least one active ingredient; and

some water.

The first surfactant is generally an anionic surfactant which can be used in the context of the invention and is advantageously chosen from carboxylates, acylamino acids, acylamino acid salts, amidoethercarboxylates, alkyl polyaminocarboxylates, alkyl ether sulfates, alkyl sulfonates, isethionates, alkyl methyltaurates, alkyl sulfosuccinates, alkyl sulfoacetates, alkyl phosphates (mono or dialkyl phosphates), and mixtures thereof.

As anionic surfactant, mention may advantageously be made of sodium or ammonium lauryl ether sulphate, sodium or ammonium lauryl sulphate, lauryl glycol carboxylate, cocoampho(di)acetate, lauroampho(di)acetate, lauryl potassium phosphate, and mixtures thereof. Preferably, the anionic surfactant is sodium lauryl ether sulphate.

Preferably, the amount of the first surfactant in the composition according to the invention is between 5 and 20% by weight relative to the total weight of the composition.

The second surfactant is generally a zwitterionic surfactant which can be used in the context of the invention and is advantageously chosen from betaine derivatives including amidopropyl betaines, amphoacetates, amphodiacetates, hydroxylsultaines, and mixtures thereof.

As derivatives of betaines, mention may be made, for example, of cocoyl betaine, lauroyl betaine, cocamidopropyl betaine, and lauramido propyl betaine. Preferably the second zwitterionic surfactant is cocamidopropyl betaine.

Preferably, the amount of the second surfactant in the composition according to the invention is between 0.1 and 10% by weight relative to the total weight of the composition.

The hair and/or skin care composition according to the invention preferably comprises at least one silicone agent. Silicone agents are often used in hair care compositions because they give suppleness and shine to the hair. They are generally in the form of particles which will be deposited around the hair, thus creating a protective layer. The silicones used for hair products are modular ingredients, which adapt to the different care products and the needs of the hair. They come in the form of chains, nets or rings. They can be both hydrosoluble and hydrophobic.

The silicone agents that may be used in accordance with the invention may be in the form of oils, waxes, resins or gums.

The silicone compound(s) that may be used in the context of the hair and/or skin care composition according to the invention may be chosen from the group comprising polydialkylsiloxanes, in particular polydimethylsiloxanes (PDMS), and organo-modified polysiloxanes comprising at least one functional group chosen from amine groups, aryl groups and alkoxy groups.

Silicone agents are defined in more detail in the work by Walter Noll “Chemistry and Technology of Silicones” (1968), Academie Press.

Preferably, the amount of silicone agents in the composition is between 0 and 10% by weight relative to the total weight of the composition.

The composition according to the invention may also contain one or more active ingredients conventionally used in care products.

Within the meaning of the invention, the term “active” is understood to mean a compound having advantageous properties (care, softening, detangling, etc.) and used in a conventional manner by a person skilled in the art.

Mention may be made, for example, of the usual hydrosoluble or liposoluble active ingredients in the cosmetics field, such as preservatives, humectants (glycerin), sequestrants (EDTA), antioxidants, perfumes, coloring materials, soluble dyes, pigments encapsulated or not, nacres, fillers with a mattifying effect, tensors, whitening agents, exfoliating agents, sunscreens, hydrophilic or lipophilic cosmetic or dermatological active ingredients such as hydrosoluble or liposoluble vitamins, antiseptics, anti-seborrheic agents, antimicrobials such as benzoyl peroxide, salicylic acid, triclosan, azelaic acid, niacin (vitamin PP), slimming agents such as caffeine, optical brighteners, electrolytes, agents whose purpose is to improve the cosmetic properties of the skin, or even spherical or non-spherical, porous or non-porous solid particles of all sizes.

The amounts of these various active ingredients are those conventionally used in the field considered, and represent for example, from 0 to 20% by weight relative to the total weight of the composition. These active ingredients and their concentration must be such that they do not modify the property sought for the composition of the invention.

The composition according to the invention generally comprises water as preparation solvent. In general, the amount of water is between 50 and 99% by weight relative to the total weight of the composition, preferably between 60 and 95%, even more preferably between 70 and 90%.

In addition to water, the composition according to the invention may comprise one or more hydrosoluble solvents chosen from lower alcohols (mono-alcohols) comprising from 1 to 8 carbon atoms, such as ethanol, isopropanol, tert-butanol, n-butanol; and polyols such as glycerin, propylene glycol, butylene glycol, isoprene glycol, glycols such as PEG-8; sorbitol; polyol ethers; sugars such as glucose, fructose, maltose, lactose, and sucrose; insofar as these compounds do not alter the properties sought from the composition according to the invention.

The amount of this (these) solvent(s) in the composition of the invention is advantageously between 0 and 30% by weight relative to the total weight of the composition, preferably between 1 and 10%.

A person skilled in the art will know how to adjust the amounts of each component of the composition in such a way that the sum of the amounts is equal to 100% by weight during its composition according to the invention.

The composition according to the invention may be in various more or less thickened forms, in particular in the form of a thickened solution, gel, milk, lotion or cream. Preferably, the composition according to the invention is in the form of a thickened solution or a gel.

The composition according to the invention is produced according to techniques known to a person skilled in the art. The latter will also be able to implement the latex according to the invention and the other ingredients in order to obtain a conventional composition for hair and/or skin care.

The composition according to the invention preferably comprises at least one solid agent in suspension.

The composition according to the invention may be used in the form of shampoo, sun cream, sun lotion, sunscreen, hand gel, hand lotion, liquid soap, shaving cream, lotion after shave, revitalizing gel, revitalizing lotion, hair dye, perm lotion, perm gel, hair relaxer, hair bleach, styling gel or shower gel. Preferably, the composition according to the invention is a shampoo composition, a cleansing composition for the skin, or a shower gel composition.

Uses and Advantages of the Invention

The latices according to the invention are advantageously used to formulate compositions for hair and/or skin care. They offer a better compromise in terms of rheological performance and transparency, including at acidic pH (3.5 to 5.5), than the latices of the prior art.

The crosslinked polymer latex according to the invention is capable of maintaining a solid agent in suspension in the composition for hair and/or skin care.

The invention also relates to a method for thickening a hair and/or skin care composition having a pH of between 3.5 and 5.5, comprising the use of the latex according to the invention as described above.

The invention and the resulting advantages will emerge better from the following examples given in order to illustrate the invention, and not in a limiting manner.

### EXAMPLES

Glossary

AMA: MethAcrylic Acid

AE: Ethyl Acrylate

ABu: Butyl Acrylate

AEH: EthylHexyl Acrylate

ABe: Poly(ethylene oxide) behenyl acrylate (25 ethoxylated units)

MBA: N-MethyleneBisAcrylamide

TMPTA: TriMethylol Propane TriAcrylate

APE: Allyl Penta Erythritole

TAAC: TretraAllyl Ammonium Chloride

SLES: Sodium Lauryl Ether Sulfate

NaOH: Sodium Hydroxide

CAPB: CocAmidoPropyl Betaine

1/ Synthesis of crosslinked polymer latices

Examples I1 to I15 and comparative examples C1 to C12 are synthesized according to the following protocol. The amounts of monomers are adjusted in order to comply with the ratios specified in Table 1 and while maintaining an amount of crosslinked polymer of 30% by weight relative to the total weight of the latex.

Latex Synthesis Protocol

423.0 g of deionized water and 2.5 g of a 30% by weight solution of sodium dodecyl sulphate are introduced into a jacketed reactor equipped with mechanical stirring and a condenser. The medium is heated to 89° C.

A pre-emulsion containing 154.0 g of deionized water, 7.5 g of a 30% by weight solution of sodium dodecyl sulfate, 90.0 g (1.05 mol) of methacrylic acid, 135 g (1.35 mol) of ethyl acrylate, 75.0 g (0.59 mol) of butyl acrylate and 0.6 g (3.9 mmol) of N-methylene-bis-acrylamide are added in this order under magnetic stirring for 10 minutes (solution A).

70.1 g of a solution of sodium persulfate at 850 ppm in deionized water is prepared (solution B).

When the reactor is at the set temperature, 28.1 g of a 4250 ppm solution of sodium persulfate in deionized water are injected into the reactor and the 2 solutions A and B are added respectively to 1.2 mL/min and 5.4 L/h for 20 minutes, then the flow rate is increased to 2.4 mL/min and 10.3 L/h respectively until the solutions are consumed.

When the additions of solutions A and B are completed, the medium is left under stirring at 84° C. for 30 minutes then cooled to 70° C. 7.4 g of a 5.1% solution of terbutyl hydroperoxide in deionized water are then added to the reactor. 10 minutes after this addition, 7.3 g of a 4.1% solution of isoascorbic acid are added in the deionized water.

After 30 minutes, the latex is filtered at 300 μm.

TABLE 1
Composition of crosslinked polymers of latices

| | Monomers | | | | | | Crosslinking agents | | | |
|---|---|---|---|---|---|---|---|---|---|---|
| | AMA | ABe | AE | ABu | AEH | AA | MBA | TMPTA | APE | TAAC |
| I1 | 35 | | 65 | | | | 0.2 | | | |
| I2 | 35 | | 55 | 10 | | | 0.2 | | | |
| I3 | 35 | | 55 | | 10 | | 0.2 | | | |
| I4 | 25 | | 55 | 20 | | | 0.2 | | | |
| I5 | 24 | | 60 | 16 | | | 0.2 | | | |
| I6 | 28 | | 62 | 10 | | | 0.2 | | | |
| I7 | 40 | | 55 | 5 | | | 0.2 | | | |
| I8 | 30 | | 45 | 25 | | | 0.2 | | | |
| I9 | 25 | | 70 | 5 | | | 0.2 | | | |
| I10 | 30 | | 65 | | | 5 | 0.2 | | | |
| I11 | 30 | | 55 | 10 | | 5 | 0.2 | | | |
| I12 | 30 | | 55 | | 10 | 5 | 0.2 | | | |
| I13 | 25 | | 65 | | | 10 | 0.2 | | | |
| I14 | 25 | | 55 | 10 | | 10 | 0.2 | | | |
| I15 | 25 | | 55 | | 10 | 10 | 0.2 | | | |
| C1 | 35 | | 65 | | | | | 0.4 | | |
| C2 | 35 | | 65 | | | | | | | 0.66 |
| C3 | 35 | | 65 | | | | | | 0.05 | |
| C4 | 35 | | | 65 | | | 0.2 | | | |
| C5 | 51 | | 49 | 1 | | | 0.2 | | | |
| C6 | 35 | | 55 | 10 | | | | 0.3 | | |
| C7 | 18 | | 50 | 32 | | | 0.2 | | | |
| C8 | 35 | 8 | 57 | | | | 0.2 | | | |
| C9 | 35 | | 65 | | | | 0.03 | | | |
| C10 | 35 | | 65 | | | | 0.6 | | | |
| C11 | 28 | | 62 | 10 | | | 0.03 | | | |
| C12 | 28 | | 62 | 10 | | | 0.6 | | | |

All polymerizations lead to stable latices with the exception of comparative examples C2 and C4.

2/ Formulation of Composition for Hair and/or Skin Care

The latices of polymers of examples I1-I15 and comparative examples C1-C12 are used to formulate a composition for hair and/or skin care. Table 2 describes the composition of the formulation. The amounts of the ingredients are given as a percentage of active ingredient.

TABLE 2
Composition for hair and/or skin care (Sa = sufficient amount)

| Ingredients | % by weight |
|---|---|
| Water | 84.5 |
| Polymer | 2.4 |
| SLES | 11.2 |
| NaOH | Sa pH 6.6 |
| CAPB | 1.9 |
| Citric acid | Sa pH 5.5 or 4.5 |

The composition contains 2.4% by weight of polymer, i.e., 8% by weight of latex.

3/ Formulation Analysis

The formulations are evaluated according to three criteria: their Brookfield viscosity at 20 rpm, their transmittance at 500 nm and their threshold stress.

The viscosity measurement is carried out at 25° C. on a Brookfield RVT viscometer equipped with a module 4. A viscosity value of between 3000 and 8000 cP (centipoise) is sought and desired in order to maintain adequate flow properties.

The transmittance of the formulation is measured on a Hach Lange DR 600 spectrophotometer at 500 nm in 1.5 mL polystyrene (PS) cups (10-mm optical path), at 25° C. The formulation is considered to be transparent when the transmittance value is greater than 85%.

The threshold stress measurement is carried out at 25° C. on a Malvern Kinexus Pro+ rheometer equipped with a cone-plane geometry (2°/60 mm). A stress rise programming from 0.1 to 100 Pa is applied and ten acquisitions are carried out per logarithmic decade. The formulation samples are centrifuged beforehand for 5 minutes at 5000 rpm in order to remove the air bubbles. This value reflects the suspensivity of the formulations and is considered sufficient and satisfactory beyond 1.5 Pa.

A result for a composition is considered good and satisfactory when the three criteria are satisfied for at least one of the tests, either at pH=5.5 or at pH=4.5.

The results are summarized in Table 3 below:

TABLE 3
Results of the analyzes

| | Brookfield | | Threshold | | Transmittance | |
|---|---|---|---|---|---|---|
| | viscosity (cP) | | stress (Pa) | | (%) | |
| | pH 5.5 | pH 4.5 | pH 5.5 | pH 4.5 | pH 5.5 | pH 4.5 |
| I1 | 3200 | 4100 | 2.12 | 2.38 | 88.1 | 87.5 |
| I2 | 4100 | 6850 | 2.49 | 2.61 | 93 | 92.8 |
| I3 | 1900 | 3600 | 1.68 | 1.99 | 93 | 93.4 |
| I4 | 5900 | 12600 | 1.98 | 2.38 | 95.1 | 92.6 |
| I5 | 2500 | 7800 | 1.57 | 3.36 | 97.2 | 96.9 |
| I6 | 4200 | 7500 | 2.67 | 3 | 94.8 | 94.4 |
| I7 | 2200 | 3300 | 1.54 | 1.93 | 87.1 | 85.9 |
| I8 | 7300 | 18600 | 1.89 | 2.38 | 96.2 | 93.8 |
| I9 | 4700 | 8000 | 2.61 | 2.93 | 94 | 93.2 |
| I10 | 3400 | 4200 | 1.8 | 2.0 | 86.2 | 85.8 |
| I11 | 4200 | 7100 | 2.1 | 2 3 | 89.8 | 89.2 |
| I12 | 2000 | 4200 | 1.6 | 1.8 | 90.4 | 90.1 |
| I13 | 3600 | 4400 | 1.6 | 1.7 | 85.7 | 85.2 |
| I14 | 4300 | 7100 | 1.8 | 1.9 | 87.3 | 86.9 |
| I15 | 4500 | 4700 | 1.5 | 1.6 | 87.9 | 87.6 |
| C1 | 2500 | 3100 | 1.25 | 1.22 | 88.7 | 88.7 |
| C2 | N/A | N/A | N/A | N/A | N/A | N/A |
| C3 | 2000 | 2600 | 1.37 | 1.47 | 86.5 | 85.6 |
| C4 | N/A | N/A | N/A | N/A | N/A | N/A |
| C5 | 2000 | 2200 | 2.03 | 1.79 | 61.9 | 51.9 |
| C6 | 1600 | 2500 | 0.62 | 0.73 | 94.2 | 93.8 |
| C7 | 5200 | 4200 | 2.23 | 1.65 | 51.1 | 42.7 |
| C8 | 11000 | 11200 | 4.9 | 5.6 | 79.4 | 76..8 |
| C9 | 1100 | 1200 | 0.3 | 0.4 | 98.9 | 98.6 |
| C10 | 8500 | 13700 | 3.8 | 4.6 | 37.6 | 33.2 |
| C11 | 1300 | 1500 | 0.2 | 0.5 | 99.6 | 99.4 |
| C12 | 9200 | 12800 | 4.1 | 5.2 | 27.5 | 25.8 |

N/A: Not Applicable because counter-examples C2 and C4 do not lead to stable latices.

The results of the examples I1 to I15 demonstrate that the latices according to the invention make it possible to satisfy the three criteria of viscosity, transmittance and threshold stress for at least one of the two pH values, whereas the latices of the comparative examples do not allow it. The latices according to the invention display, for at least one of the pH values, both a viscosity of between 3000 and 8000 cP, a transmittance greater than 85% and a threshold stress greater than 1.5 Pa. On the contrary, none of the counter-examples C1-C8 meets these three criteria for the same pH value. Therefore, the latices according to the invention offer a better compromise in terms of rheological properties and transparency.

Furthermore, it is observed that the latices according to the invention containing acrylic acid (I10 to I15) offer a satisfactory compromise in terms of rheological properties and transparency. This compromise is improved even more markedly when the respective latices contain more methacrylic acid, and even more so when they do not contain acrylic acid, which results, in particular, in a lower threshold stress and transmittance. This is appreciable by comparing in particular the latices I1, I2, and I3 with the latices I10, I11 and I12 respectively, and I13, I14 and I15 respectively, the latter having been obtained by replacing 5% or 10% of methacrylic acid by acrylic acid in the former. This compromise becomes more advantageous as the amount of methacrylic acid increases, i.e., when the amount of acrylic acid decreases.

Latices C9 to C12 are similar to latices I1 and I6, but differ in their amount of MBA crosslinking agent and respectively equal to 0.03% by weight (300 ppm) and 0.6% by weight (6000 ppm). It is observed that the best results are obtained when the MBA concentration is between 0.05% by weight (500 ppm) and 0.5% by weight (5000 ppm). In this case (500-5000 ppm of MBA), the latices obtained provide the best compromise in terms of rheological properties and transparency.

4/ Evaluation of the Latices of the Invention Compared to the Comparative Examples.

The latices of examples I1, I2, I6 and I9 are compared with the examples deemed relevant from the patents cited in the prior state of the art (Table 4). Latices are used in the formulation described previously and the same parameters were studied.

TABLE 4
Comparative examples C13 to C15 of the prior art

| Comparative example | Prior art | Example |
|---|---|---|
| C13 | US 2013/0112185 | Example 1 |
| C14 | WO 2015/158668 | Example 7 |
| C15 | WO 2016/102790 | Example 6 |

The results are recorded in Table 5 below.

TABLE 5
Test results

| | Brookfield | | Threshold | | Transmittance | |
|---|---|---|---|---|---|---|
| | viscosity (cP) | | stress (Pa) | | (%) | |
| | pH 5.5 | pH 4.5 | pH 5.5 | pH 4.5 | pH 5.5 | pH 4.5 |
| I1 | 3200 | 4100 | 2.12 | 2.38 | 88.1 | 87.5 |
| I2 | 4100 | 6850 | 2.49 | 2.61 | 93 | 92.8 |
| I6 | 4200 | 7500 | 2.67 | 3 | 94.8 | 94.4 |
| I9 | 4700 | 8000 | 2.61 | 2.93 | 94 | 93.2 |
| C13 | 2200 | 2500 | 0.97 | 1.19 | 82.6 | 78.6 |
| C14 | 9100 | 10300 | 1.82 | 2.34 | 84.2 | 82.8 |
| C15 | 8300 | 8600 | 1.77 | 2.44 | 83.4 | 83.7 |

The results of the tests show that the polymers of comparative examples C13 to C15 do not make it possible to achieve levels of transmittance as high as those of the polymers of the invention. The C13 latex polymer, having the same composition as that of example I1 but crosslinked with TMPTA, does not make it possible to achieve properties as advantageous as when the polymer is crosslinked with MBA. The polymers of comparative examples C14 and C15, which contain monomer units having an alkyl chain of at least 10 carbon atoms, or monomer units of formula —(CHR¹—CHR²—O)— with R¹ and R² being, independently of each other, a hydrogen atom or an alkyl group, offer levels of transmittance lower than those obtained with the latices according to the invention.

1. A latex comprising a crosslinked polymer, said polymer comprising:
from 20 to 45% by weight of monomer unit of (meth)acrylic acid;
from 35 to 78% by weight of monomer unit of methyl acrylate and/or ethyl acrylate; and
from 0 to 30% by weight of monomer unit of at least one linear alkyl acrylate, the linear alkyl comprising 3 to 9 carbon atoms;
the sum of the monomer units being equal to 100% by weight;
wherein:
the polymer is crosslinked with N-methylene-bis-acrylamide;
and in that the polymer is free of monomer unit having an alkyl chain of at least 10 carbon atoms, and monomer unit of formula —(CHR¹—CHR²—O)—, R¹ and R² being, independently of each other, a hydrogen atom or an alkyl group.

2. The latex according to claim 1, wherein the polymer is crosslinked with an amount of N-methylene-bis-acrylamide of between 500 and 5000 ppm by weight relative to the total weight of monomers.

3. The latex according to claim 1, wherein the polymer is crosslinked with N-methylene-bis-acrylamide as sole crosslinking agent.

4. The latex according to claim 1, wherein the amount of monomer unit of (meth)acrylic acid is between 25 and 40% by weight.

5. The latex according to claim 1, wherein the polymer comprises an amount of monomer unit of acrylic acid of less than 20% by weight.

6. The latex according to claim 1, wherein the polymer is free of monomer unit of acrylic acid.

7. The latex according to claim 1, wherein the crosslinked polymer comprises:
from 20 to 45% by weight of monomer unit of methacrylic acid;
from 35 to 78% by weight of monomer unit of ethyl acrylate; and
from 0 to 30% by weight of monomer unit of at least one linear alkyl acrylate, the alkyl comprising 3 to 9 carbon atoms.

8. The latex according to claim 1, wherein the amount of linear alkyl acrylate monomer unit, the linear alkyl comprising 3 to 9 carbon atoms, is between 1 and 25% by weight.

9. The latex according to claim 1, wherein the linear alkyl acrylate, the linear alkyl comprising 3 to 9 carbon atoms, is chosen from butyl acrylate, ethylhexyl acrylate and mixtures thereof.

10. The latex according to claim 1, wherein the latex comprises between 20 and 40% by weight of crosslinked polymer, between 60 and 80% by weight of water, and between 0.1 and 2% of surfactant compounds and/or post-treatment compounds, relative to the weight of the latex.

11. The latex according to claim 1, wherein the latex is free of monomer unit of 2-acrylamido-2-methylpropanesulfonic acid.

12. The latex according to claim 1, wherein the latex is free of a strong acid monomer unit.

13. A hair and/or skin care composition comprising at least one latex according to claim 1.

14. The composition for hair and/or skin care according to claim 13, wherein the composition also comprises:
at least a first surfactant;
at least one second surfactant different from the first surfactant;
optionally at least one silicone agent;
optionally at least one active ingredient; and
some water.

15. A method for thickening a hair and/or skin care composition having a pH of between 3.5 and 5.5, said method comprising preparing the hair and/or skin care composition such that it comprises the latex according to claim 1.

16. The latex according to claim 2, wherein the polymer is crosslinked with N-methylene-bis-acrylamide as sole crosslinking agent.

17. The latex according to claim 16, wherein the amount of monomer unit of (meth)acrylic acid is between 25 and 40% by weight.

18. The latex according to claim 16, wherein the polymer comprises an amount of monomer unit of acrylic acid of less than 5% by weight.

19. The latex according to claim 16, wherein the crosslinked polymer comprises:
from 20 to 45% by weight of monomer unit of methacrylic acid;
from 35 to 78% by weight of monomer unit of ethyl acrylate; and
from 0 to 30% by weight of monomer unit of at least one linear alkyl acrylate, the alkyl comprising 3 to 9 carbon atoms.

20. The latex according to claim 19, wherein:
the linear alkyl acrylate, the linear alkyl comprising 3 to 9 carbon atoms, is chosen from butyl acrylate, ethylhexyl acrylate and mixtures thereof;
the amount of the linear alkyl acrylate monomer unit, the linear alkyl comprising 3 to 9 carbon atoms, is between 1 and 25% by weight; and
the latex comprises between 20 and 40% by weight of crosslinked polymer, between 60 and 80% by weight of water, and between 0.1 and 2% of surfactant compounds and/or post-treatment compounds, relative to the weight of the latex.
